(19) United States
(12) Patent Application Publication
(10) Pub. No.: US 2016/0160854 A1
(43) Pub. Date: Jun. 9, 2016

(54) ROTARY-WAVE SUB-ASSEMBLY FOR PUMPING A FLUID AND ROTARY-WAVE PUMPING DEVICE

(71) Applicant: EVEON, Montbonnot Saint Martin (FR)

(72) Inventors: Christophe Dehan, Saint Ismier (FR); Arnaud Wattellier, Eybens (FR)

(21) Appl. No.: 14/906,590

(22) PCT Filed: Jun. 11, 2014
(86) PCT No.: PCT/FR2014/051413

§ 371 Date: Jan. 21, 2016

(30) Foreign Application Priority Data
Jul. 22, 2013 (FR) 1357189

(51) Int. Cl. F04B 49/22; F04B 9/04; A61M 1/00; F04B 53/16; F04B 53/10; F04B 19/22; F04B 53/14

(52) U.S. Cl. CPC F04B 49/22; F04B 19/22; F04B 9/042; F04B 53/14; F04B 53/16; F04B 53/10; A61M 1/00

(57) ABSTRACT

A rotary reciprocating sub-assembly for pumping a fluid, the sub-assembly comprises: a hollow body provided with two ducts; and a piston that co-operates with the body to define a working chamber, the piston being movable angularly between two fluid-flow configurations and switching configurations, and being movable in reciprocating longitudinal translation so as to cause the dimensions of the working chamber to vary, the ducts are longitudinally offset from each other, the piston includes a channel that opens out longitudinally into the working chamber and radially into a pair of side recesses that are longitudinally offset, and that are arranged so that, in each fluid-flow configuration, only one of the recesses is in fluid-flow communication with only one of the ducts.

### TECHNICAL FIELD

The invention generally relates to a rotary reciprocating sub-assembly for pumping a fluid and to a rotary reciprocating pumping device for positive displacement pumping of a fluid.

### PRIOR ART

The use of positive displacement pumping devices is known for producing and/or reconstituting mixtures (liquid-solid or liquid-liquid mixtures) and/or administering fluids (by injection, infusion, orally, spray, . . . ), in particular for medical, cosmetic, veterinary applications. For these types of application, accurate quantities of fluid need to be pumped in controlled manner, but it is also often necessary to connect the fluid inlet and outlet of the pumping device to additional fluid-flow devices, e.g. a fluid-flow dispenser that makes it possible to transfer the fluid to one or more administering containers or devices in order to perform a plurality of fluid-flow functions with a single pump.

Known pumping devices generally present ducts that are arranged at 180° to each other. Also, such pumping devices are connected in fluid-flow manner with other fluid-flow devices by means of flexible or rigid pipes that are bulky and that risk catching and being pulled off during handling. Pumping devices are thus voluminous and awkward to use, which is prejudicial to their use, in particular for applications in the medical field. Such a pumping device is known from U.S. Pat. No. 3,168,872, which describes a rotary reciprocating pumping device comprising a hollow body defining a cavity and having a wall with two ducts passing therethrough, opening out into the cavity, and arranged at 180° to each other. That rotary reciprocating pumping device also comprises a piston housed in the cavity in which it is movable angularly and in reciprocating axial translation so as to vary the dimensions of the working chamber that it defines together with the cavity. The piston includes a flat that is suitable for being successively in communication with one, then none, then the other one of the ducts. Thus, the fluid may be sucked in via one of the ducts, stored in the working chamber, and then discharged via the other duct.

### SUMMARY OF THE INVENTION

The object of the invention is to remedy those drawbacks by proposing a rotary reciprocating pumping sub-assembly and a rotary reciprocating pumping device that allow a relative angular orientation of the intake and discharge ducts that is different from 180°, of manufacturing cost that is moderate, with a limited number of parts, that are reversible, that are accurate, that make it possible to transfer viscous liquid even at high pressure, and that have good fluid-flow and energy efficiency.

To this end, the invention provides a rotary reciprocating sub-assembly for pumping a fluid, in particular for medical use, said sub-assembly comprising: a hollow body defining a cavity of longitudinal axis and having a wall with two ducts passing therethrough and opening out radially into said cavity; and a piston housed in said cavity with which it co-operates to define a working chamber, the piston being movable angularly between fluid-flow configurations in which it allows fluid-flow communication between the working chamber and one of the ducts, and switching configurations in which it prevents any fluid-flow communication with each of the ducts, the piston being movable in reciprocating longitudinal translation so as to cause the dimensions of the working chamber to vary and successively suck in and then discharge the fluid via one and then the other of the ducts, said sub-assembly being characterized in that the ducts are longitudinally offset from each other, in that the piston includes a channel that opens out longitudinally into the working chamber and radially into at least one pair of side recesses that are provided in the periphery of the piston, that are longitudinally offset from each other, and that are arranged so that, in each fluid-flow configuration, only one of the recesses is in fluid-flow communication with only one of the ducts, in that the recesses of the pair are angularly offset from each other by an angle that is different from 180°, and in that the ducts are angularly offset from each other by the same angle.

The idea on which the invention is based is to provide the recesses of the piston at different heights so that they can be angularly oriented in any good configuration for putting the ducts of the body in fluid-flow connection.

The rotary reciprocating sub-assembly of the invention may advantageously present the following features:
the recesses are superposed in a longitudinal plane, and the ducts are superposed in a longitudinal plane;
the piston includes n pairs of recesses distributed in two disjoint radial planes, and the channel is arranged to open out radially into the n recesses such that during one complete revolution of the piston in the body, the piston is n times in each of the fluid-flow configurations;
it includes at least first and second levels, each corresponding, in distinct manner, to a set of two ducts, to a working chamber, to a channel, and to a pair of recesses;
the working chambers present sections that are different;
it includes at least a cam and a guide finger, one carried by the piston, the other by the body, and arranged to co-operate reciprocally so that turning the piston relative to said body causes:
   over a first angular portion, the piston to move in axial translation relative to the body in a first direction;
   over a second angular portion, the piston to be axially stationary relative to the body;
   over a third angular portion, the piston to move in axial translation relative to the body in a second direction; and
   over a fourth angular portion, the piston to be axially stationary relative to the body;

the ducts and the recesses being arranged so that the ducts are closed during the second and fourth angular portions.

The invention extends to a rotary reciprocating fluid pumping device, in particular for medical use, the device being characterized in that it comprises drive means and a rotary reciprocating sub-assembly for pumping a fluid as described above, and releasable mechanical coupler means for mechanically connecting said drive means to said piston in releasable manner.

The rotary reciprocating pumping device may include a rotary reciprocating sub-assembly for pumping a fluid, the device including at least first and second levels, each corresponding, in distinct manner, to a set of two ducts, to a working chamber, to a channel, and to a pair of recesses; the working chambers presenting sections that are different, one of the ducts is connected to the inlet for a first fluid, one of the ducts is connected to the inlet for a second fluid, the rotary reciprocating pumping device includes mixer means for mixing the first fluid discharged by the other of the ducts, and the second fluid discharged by the other of the ducts, the resulting mixture being proportional to the difference in section between the working chambers.

### BRIEF DESCRIPTION OF THE DRAWINGS

The present invention can be better understood and other advantages appear on reading the detailed description of two embodiments given by way of non-limiting example and shown in the accompanying drawings, in which:

FIG. 1 is a side view of the piston of the rotary reciprocating pumping sub-assembly in a first embodiment of the invention;

FIGS. 2 to 7 are transparent side views of the rotary reciprocating sub-assembly in the first embodiment of the invention, shown in six distinct operating positions during a pumping cycle (intake, switching, discharge, switching);

FIG. 8 is a section view of the body and of the piston of the rotary reciprocating pumping sub-assembly in a second embodiment of the invention; and

FIGS. 9 to 14 are section views of the rotary reciprocating sub-assembly in the second embodiment of the invention, the piston not being shown in section, the rotary reciprocating sub-assembly being shown in six distinct operating positions during a pumping cycle.

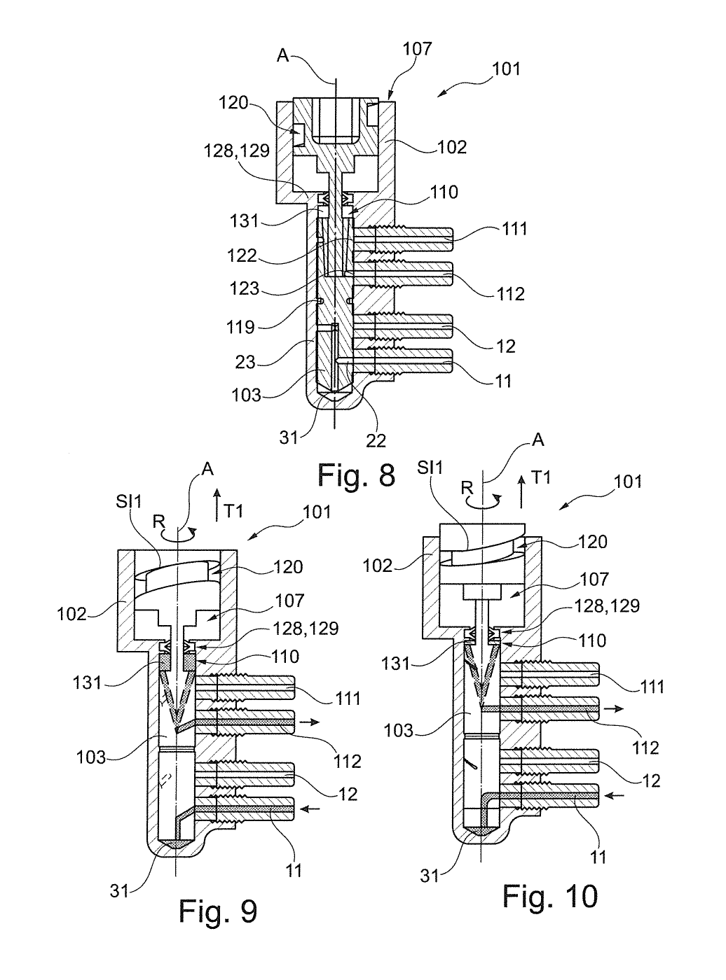

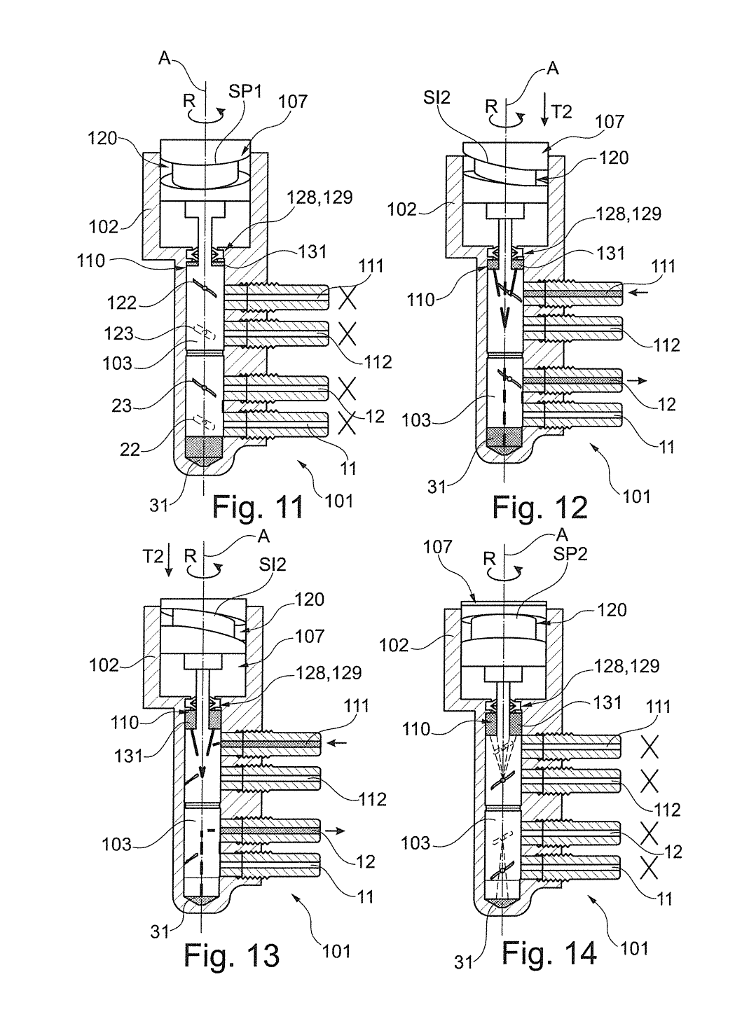

In FIGS. 8 to 14, elements that are similar to elements in the preceding figures are given the same reference numbers, plus 100.

### DESCRIPTION OF THE EMBODIMENTS

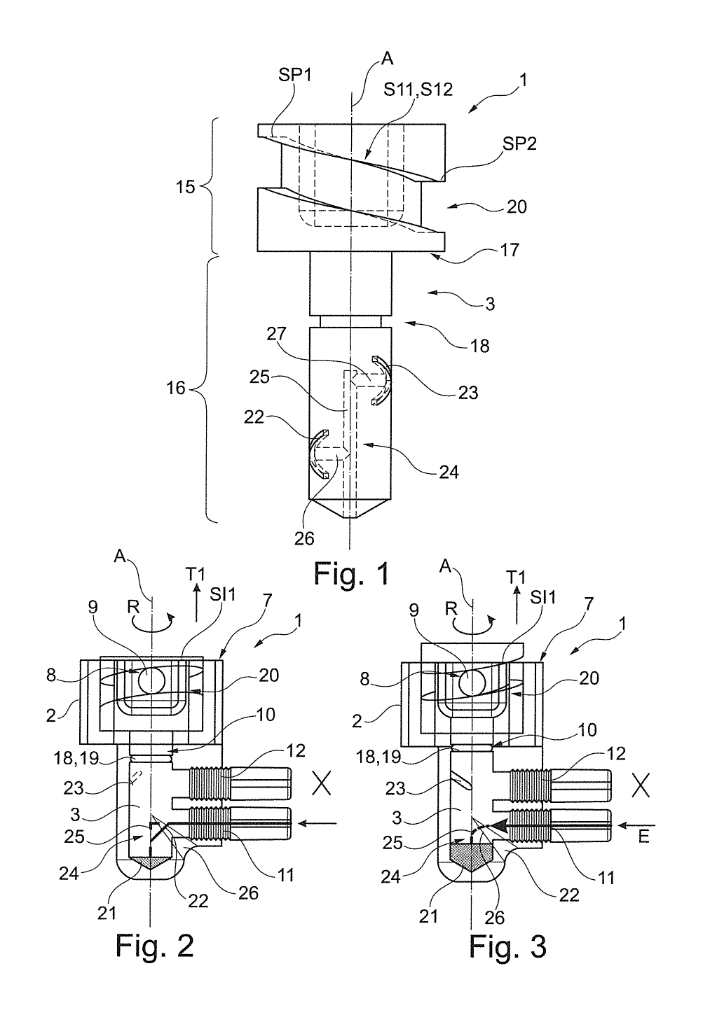

The rotary reciprocating pumping sub-assembly of the invention may present a single-acting configuration having a single level, described below as a first embodiment shown in FIGS. 1 to 7, or a multi-acting configuration having a plurality of levels, e.g. the double-acting configuration described below as a second embodiment shown in FIGS. 8 to 14.

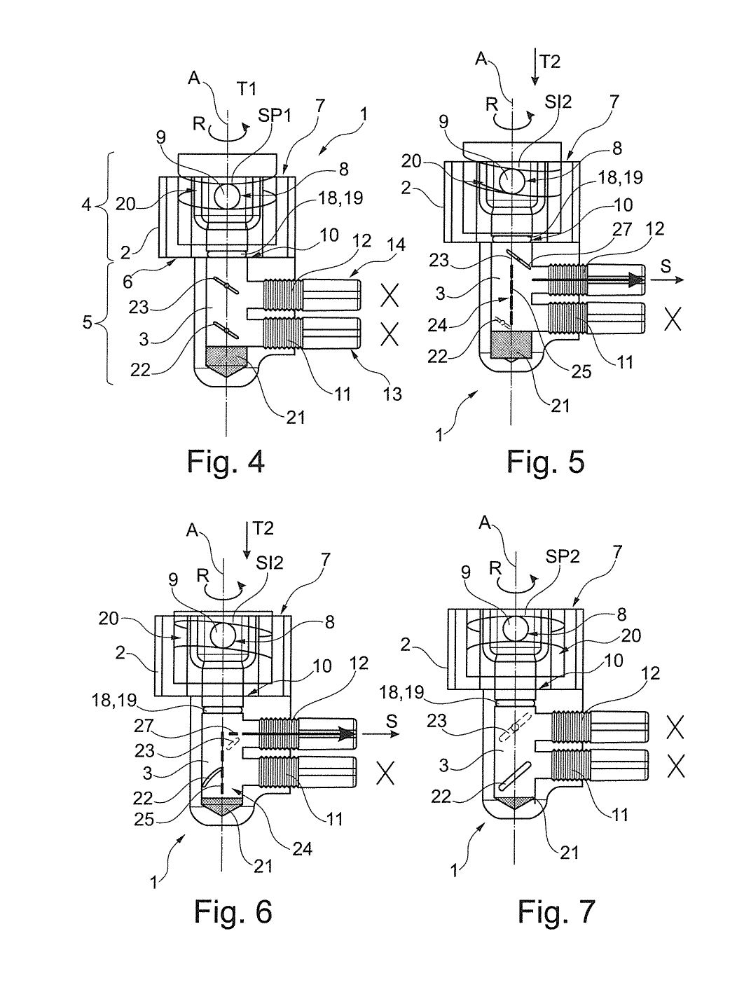

With reference to FIGS. 2 to 7, the rotary reciprocating sub-assembly 1 in the first embodiment of the invention comprises a body 2, a piston 3, and a guide finger 9.

The body 2 is hollow and is formed of two cylindrical portions 4, 5 (shown in FIG. 4) of different diameters, connected together via a shoulder 6. By way of example, the body 2 is made of plastics material or of any other suitable material.

The inside of the larger-diameter cylindrical portion 4 forms a bore 7 of longitudinal axis A. The free end of the larger-diameter cylindrical portion 4 is open and is for receiving the piston 3 in longitudinal sliding. The other end is connected to the smaller-diameter cylindrical portion via the shoulder 6. The wall of the larger-diameter cylindrical portion 4 has an orifice 8 passing therethrough for receiving a radial guide finger 9 that is arranged so as to extend into the bore 7. By way of example, the guide finger 9 is a pin that is secured to the body by elastic deformation and/or by adhesive and/or by any other suitable means. By way of example, the guide finger 9 presents a cylindrical section or any other suitable section.

The inside of the smaller-diameter cylindrical portion 5 defines a cavity 10 of longitudinal axis A and of diameter that is less than the diameter of the bore 7. The free end of the smaller-diameter cylindrical portion 5 is closed and forms the bottom of the body 2. The bore 7 and the cavity 10 are for receiving the piston 3 housed in the body 2. The free end of the piston 3 thus co-operates with the body 2 to define a working chamber 21. The wall of the smaller-diameter cylindrical portion 5 has two ducts 11, 12 passing therethrough that are longitudinally offset from each other and that open out radially into the cavity 10. In the embodiment shown, the ducts 11, 12 are longitudinally superposed, and thus angularly offset by an angle of 0°. In another embodiment (not shown), the ducts may be angularly offset relative to each other by any other appropriate angle, e.g. 90°. By way of example, the ducts 11, 12 have a section that is circular, and they present the same diameter. The body 2 includes connector endpieces 13, 14 (shown in FIG. 4) each surrounding a respective duct 11, 12 and being suitable for connecting to a fluid-flow dispenser by means of an intake or discharge pipe (not shown). The connector endpieces 13, 14 may also be connected to the fluid-flow dispenser or incorporated directly in the fluid-flow dispenser. For the purposes of description, the terms “intake” duct 11 and “discharge” duct 12 are used below in non-limiting manner in order to distinguish between the ducts 11, 12. Naturally, depending on the selected operating configuration, each of the ducts 11, 12 may be used equally well to admit or to discharge fluid.

With reference in particular to FIG. 1, the piston 3 is made up of two cylindrical portions 15, 16 of different diameters, connected together via a shoulder 17. The smaller-diameter and larger-diameter cylindrical portions 16, 15 of the piston 3 present respective outside diameters that are slightly smaller than the diameters respectively of the cavity 10 and of the bore 7 in which the piston 3 can thus be housed. By way of example, the piston 3 is made of plastics material or of any other suitable material.

The piston 3 includes a peripheral groove 18 provided on the smaller-diameter cylindrical portion 16 for receiving a sealing gasket 19 in contact with the inside wall of the cavity 10. The sealing gasket 19 is made of a material presenting a modulus of elasticity that is less than the moduluses of elasticity of the piston 3 and the body 2. By way of example, it is made of elastomer, of silicone rubber, of rubber, of thermoplastic elastomer (TPE), or of any other suitable material.

The piston 3 also includes a groove 20 defining a dual guide cam for guiding the guide finger 9. In the longitudinal direction, the height of the groove 20 is matched to the dimensions of the guide finger 9, so as to allow it to be guided without excessive slack. The groove 20 includes first and second sloping portions SI1, SI2 that are symmetrical to each other about a longitudinal midplane. The first and second sloping portions SI1, SI2 thus present opposite slopes at the periphery of the piston 3. The first and second sloping portions SI1, SI2 are spaced apart from each other by first and second plane portions SP1, SP2 that are substantially parallel to each other and perpendicular to the longitudinal axis A. Thus, by means of the guide finger 9 and the groove 20, turning the piston 3 relative to the body 2 in a first turning direction R causes the piston 3 successively to move in axial translation relative to the body 2 in a first translation direction T1 along the first sloping portion SI1, then to be axially stationary relative to the body 2 along the first plane portion SP1, then to move in axial translation relative to the body 2 in a second translation direction T2 along the second sloping portion SI2, and then finally to be axially stationary relative to the body 2 along the second plane portion SP2, and so on. The piston 3 thus reciprocates between a high position (cf. FIG. 4) in which the working chamber 21 presents a maximum volume, and a low position (cf. FIG. 7) in which the working chamber 21 presents a minimum volume. Between the two positions of the piston 3, the working chamber 21 admits and then discharges the fluid.

The piston includes two side recesses 22, 23 that are provided in the periphery of the smaller-diameter cylindrical portion 16 of the piston 3. The recesses 22, 23 are angularly offset from each other by a second angle of 180° in this embodiment, and they are longitudinally offset by a distance that is dependent, in particular, on the profile of the groove 20 which is designed so that, at any time, only one at most of the recesses 22, 23 faces only one of the ducts 11, 12. When the ducts 11, 12 are offset from each other by an angle that is different from 0°, the recesses 22, 23 are angularly and longitudinally offset correspondingly, also taking account of the profile of the groove 20. For the purposes of description, the terms “intake” recess 22 and “discharge” recess 23 are used below in non-limiting manner in order to distinguish between the recesses 22, 23. Naturally, depending on the selected operating configuration, each of the recesses may be used equally well to admit or to discharge fluid.

The piston includes a channel 24, also provided in the smaller-diameter cylindrical portion 16. With reference to FIG. 1, the channel 24 comprises a longitudinal segment 25 that opens out longitudinally into the working chamber 31, and two radial segments 26, 27 each opening out radially into a respective one of the recesses 22, 23.

In the embodiment shown, each of the recesses 22, 23 presents the shape of a sloping groove. The grooves 22, 23 slope in opposite directions over the developed surface of the piston 3. The slope of the grooves 22, 23 is directly associated with the direction of the slope of the groove 20, such that during each of the intake and discharge stages, the channel 24 opens via one of the recesses 22, 23, in fluid-flow communication respectively with the corresponding intake duct 11 or discharge duct 12. Thus, the sloping grooves 22, 23 correspond to the minimum profile of the recesses, making it possible to limit the dead volume of the rotary reciprocating sub-assembly 1. In other embodiments that are not shown, the recesses 22, 23 may present a flat or any other suitable profile, for example.

By way of example, the free end of the larger-diameter cylindrical portion 15 of the piston 3 presents a recessed shape (not shown) for mechanically connecting the piston 3 to rotary drive means (not shown).

The single-acting rotary reciprocating sub-assembly 1 is thus provided with a single level comprising two ducts 11, 12, a working chamber 31, a channel 24 with a longitudinal segment 25 and two radial segments 26, 27, and two recesses 22, 23. Thus, a pair of recesses 22, 23 corresponds to an “intake” and “discharge” pair of ducts 11, 12.

In an embodiment that is not shown, the piston includes n pairs of recesses, each pair comprising an intake recess situated in the same radial plane as the above-described intake recess, and a discharge recess situated in the same radial plane as the above-described discharge recess. The n intake recesses are angularly arranged in manner similar to the n discharge recesses. Naturally, the n intake recesses are angularly offset relative to the n discharge recesses so that each intake configuration does not correspond to a discharge configuration and vice versa. Thus, during one complete revolution of the piston in the body, the piston is n times in an intake configuration and n times in a discharge configuration. Between each intake and discharge configuration, the piston is in an intermediate position that corresponds to a switching configuration. In order to guarantee such operation, the cam presents n first and second sloping portions that are separated from one another by plane portions.

In order to cause the single-acting rotary reciprocating sub-assembly 1 to operate, the intake duct is connected to a fluid delivery pipe (not shown), the discharge duct is connected to a fluid discharge pipe (not shown) for discharging the same fluid, and the piston 3 is mechanically connected to rotary drive means (not shown) of known type. As a result of the intake and discharge ducts 11, 12 being longitudinally superposed, it is easier to connect the rotary reciprocating sub-assembly 1 to any other fluid-flow device. Specifically, a fluid-flow dispenser may be provided in direct alignment with the intake and discharge ducts 11, 12, without requiring a pipe passing around the rotary reciprocating sub-assembly 1, or it may even be provided directly without requiring any pipe.

The operation of the single-acting rotary reciprocating sub-assembly 1 of the invention is described below with reference to FIGS. 2 to 7.

In the intake stage shown in FIGS. 2 and 3, the guide finger 9 passes along the first sloping portion SI1 of the groove 20, which transforms the turning movement R of the piston 3 into first movement in translation T1 along a first travel direction of the piston 3 relative to the body 2, which causes the piston 3 to pass from a low position (FIG. 7) in which the working chamber 31 presents a minimum volume, to a high position (FIG. 4) in which the working chamber 31 presents a maximum volume. During the first movement in translation T1, the piston 3 turns relative to the body 2, with the intake recess 22 passing in front of the orifice of the intake duct 11. Thus, the intake duct 11 is in fluid-flow communication with the working chamber 31 via the intake recess 22 and via the radial and longitudinal segments 26, 25 of the channel 24. The fluid is sucked in along arrow E by suction created by the increase in the volume of the working chamber 31. During the intake stage, the discharge recess 23 passes in front of the wall of the cavity 10, without facing the discharge duct 12. Thus, during the intake stage, the fluid does not leave the working chamber 31 via the discharge duct 12, and this is represented by a cross. The piston 3 continues to turn R relative to the body 2 until a first switching stage is reached.

In the first switching stage shown in FIG. 4, the guide finger 9 passes along the first plane portion SP1 of the groove 20. The piston 3 turning R thus does not cause it to move in translation, and the piston 3 is axially stationary in its high position. Thus, the volume of the working chamber 31 does not vary and remains at its, maximum. During the switching stage, the intake recess 22 and the discharge recess 23 each face the cavity 10 that prevents any fluid-flow communication between the working chamber 31 and one or the other of the intake or discharge ducts 11, 12. Thus, the working chamber 31 is closed in leaktight manner. The piston 3 continues to be turned R relative to the body 2 until the discharge stage is reached.

In the discharge stage shown in FIGS. 5 and 6, the guide finger 9 passes along the second sloping portion SI2 of the groove 20, which transforms the turning R of the piston 3 into a second movement in translation T2 along a second travel direction that is opposite to the first travel direction during the movement in translation T1. Thus, the piston 3 passes from its high position (FIG. 4) to its low position (FIG. 7). During the second movement in translation T2, the piston 3 turns relative to the body 2, with the discharge recess 23 passing in front of the orifice of the discharge duct 12. Thus, the discharge duct 12 is in fluid-flow communication with the working chamber 31 via the recess 23 and via the radial and longitudinal segments 27, 25 of the channel 24. The fluid is discharged via the discharge duct 12 along arrow S by pressure due to the decrease in the volume of the working chamber 31. During the discharge stage, the intake recess 22 passes in front of the wall of the cavity 10, without facing the intake duct 11. Thus, during the discharge stage, the fluid does not enter the working chamber 31 via the “intake” duct 11. The piston 3 continues to be turned R relative to the body 2 until a second switching stage is reached.

This second switching stage shown in FIG. 7 is substantially similar to the first switching stage. It differs therefrom in that the piston 3 is in the low position, the working chamber 31 presents a minimum volume, and the position of the intake and discharge recesses 22, 23 relative to the intake and discharge ducts 11, 12, is diametrally inverted relative to the first switching stage.

The rotary reciprocating cycle may be repeated. Naturally, depending on the direction of turning of the piston 3 relative to the body 2, the “intake” duct could correspond to the discharge duct and vice versa.

By modifying the profiles of the first and second sloping portions SI1, SI2 and the profiles of the intake and discharge recesses 22, 23, the ratio between the intake stage and the discharge stage may be adjusted. The duration of one of the intake and discharge stages may thus be extended relative to the duration of the other.

The rotary reciprocating sub-assembly 101 in the second embodiment of the invention is shown in FIGS. 8 to 14 and presents a double-acting configuration. To this end, it comprises two levels, a first level similar to the rotary reciprocating sub-assembly 1, and a second level comprising intake and discharge ducts 111, 112, a working chamber 131, a channel 124 that is separate from the channel 24 of the first level, and intake and discharge recesses 122, 123 like those of the first level. Thus, a pair of intake and discharge recesses 22, 23, 122, 123 corresponds to each pair of intake and discharge ducts 11, 12, 111, 112.

In the embodiment shown, the intake recesses 22, 122 are longitudinally separated by the discharge recesses 23, 123, and the intake and discharge ducts 11, 12, 111, 112 are longitudinally superposed. Furthermore, the intake recess 22 of the first level is longitudinally superposed by the discharge recess 123 of the second level, and the discharge recess 23 of the first level is longitudinally superposed by the intake recess 122 of the second level. The rotary reciprocating sub-assembly 101 thus makes it possible simultaneously to be in the intake stage for one level and in the discharge stage for the other level.

As shown in detail in FIG. 8, the channel 124 of the second level presents a frustoconical shape that flares towards the working chamber 131.

The body 102 includes a cavity 110 that presents a greater height longitudinally, thus making it possible to house both levels. In this embodiment, the sealing gasket 119 is provided between the two levels. Furthermore, the body 103 includes an annular furrow 128 that is provided between the bore 107 and the cavity 110, receiving an additional sealing gasket 129.

In a first configuration, the “intake” ducts 11, 111 of the first and second levels may be in fluid-flow connection with a common inlet for a single fluid, and the “discharge” ducts 12, 112 of the first and second levels may be in fluid-flow connection with a common outlet for a single fluid.

In a second configuration, the “discharge” duct 12, 112 of one level may be in fluid-flow connection with the “intake” duct 11, 111 of the other level.

In a third configuration, the double-acting rotary reciprocating sub-assembly may advantageously be used to create proportional doses per revolution by using one level for a first fluid and another level for a second fluid. Optionally, in order to obtain a proportional mixture, the “discharge” ducts 12, 112 of each level are connected to a single container for receiving the two fluids, for example. By modifying the ratio between the sections of the working chambers 31, 131, it is possible to vary the dosage between the two fluids proportionally, the stroke always being identical for both working chambers 31, 131.

In these three configurations, the flowrate of the pumping device incorporating such a double-acting rotary reciprocating sub-assembly 101 is increased, with a pulsation frequency that is twice that of a single-acting rotary reciprocating sub-assembly 1.

In a fourth configuration, the two levels may be identical and merely offset from each other longitudinally. Thus, the two intake stages of the two levels coincide, and the two discharge stages of the two levels coincide. In this configuration, the flowrate of the pumping device incorporating such a double-acting rotary reciprocating sub-assembly 101 is doubled, with a pulsation frequency that is identical to that of a single-acting rotary reciprocating sub-assembly 1.

The invention makes it possible to achieve the above-mentioned objects. Specifically, the rotary reciprocating sub-assembly 1, 101 of the invention makes it possible to obtain intake and discharge ducts with a relative angular orientation that is different from 180°, e.g. 0°, or any other suitable angle. If necessary, the intake and discharge ducts could also be oriented at 180°. Thus, the rotary reciprocating sub-assembly 1, 101 may easily be connected to any other fluid-flow device with a simple and direct fluid-flow circuit.

In addition, the rotary reciprocating sub-assembly 1, 101 of the invention is reversible, merely by reversing the direction in which the piston 3, 103 is turned. Thus, the “intake” duct 11, 111 becomes the “discharge” duct 12, 112 and vice versa. Mechanically uncoupling the piston 3, 103 and the drive means makes it possible to obtain a disposable rotary reciprocating sub-assembly while the motor portion is reusable. It is thus possible to guarantee that the rotary reciprocating sub-assembly 1, 101 is sterile at lower cost by replacing it between two uses. Thus, only the fluid-flow portion of the rotary reciprocating pumping device is renewed, the motor and control portions being conserved between two uses. Since the axial forces are transmitted by the cam, it is possible to use drive means that are rotary only, and to use mechanical coupler means between the piston 3 and the drive means that transmit torque only. In addition, the groove 20, 120 and the guide finger 9 make it possible to guarantee that the reciprocating movement in translation of the piston 3 is synchronous with turning of that piston 3.

The rotary reciprocating sub-assembly 1, 101 of the invention prevents any fluid flow with the “intake and discharge” ducts 11, 111, 12, 112 during the switching stages, but without creating the effect of excess pressure or suction by hydraulic blocking during these stages. Furthermore, the recesses 22, 23, 122, 123 make it possible to limit dead volume.

The contact between the sealing gasket and the body makes it possible to set the position of the rotary reciprocating sub-assembly 1, 101 angularly in the factory during its initial assembly. The angular setting is thus easily conserved until the rotary reciprocating sub-assembly 1, 101 is put into service in the rotary reciprocating device. Nevertheless, it is possible to provide a visible mark of the angular position of the piston 3, 103 relative to the body 2, 102, or a sensor of any suitable technology.

Naturally, the present invention is not limited to the above description of an embodiment, and it may be subjected to various modifications without going beyond the ambit of the invention.

1. A rotary reciprocating sub-assembly for pumping a fluid, in particular for medical use, said sub-assembly comprising:
a hollow body defining a cavity with a longitudinal axis and having a wall with two ducts passing therethrough and opening out radially into said cavity; and
a piston housed in said cavity with which it co-operates to define a working chamber, said piston being movable angularly between fluid-flow configurations in which it allows fluid-flow communication between said working chamber and one of said ducts, and switching configurations in which it prevents any fluid-flow communication with each of said ducts, said piston being movable in reciprocating longitudinal translation so as to cause the dimensions of said working chamber to vary and successively suck in and then discharge said fluid via one and then the other of said ducts,
wherein said ducts are longitudinally offset from each other,
wherein said piston includes a channel that opens out longitudinally into said working chamber and radially into at least one pair of side recesses that are provided in the periphery of said piston, that are longitudinally offset from each other, and that are arranged so that, in each fluid-flow configuration, only one of said recesses is in fluid-flow communication with only one of said ducts,
in that wherein said recesses of said pair are angularly offset from each other by an angle that is different from 180°, and
in that wherein said ducts are angularly offset from each other by the same angle.

2. The rotary reciprocating pumping sub-assembly according to claim 1, wherein said recesses are superposed in a longitudinal plane, and wherein said ducts are superposed in a longitudinal plane.

3. The rotary reciprocating pumping sub-assembly according to claim 1, wherein said piston includes n pairs of recesses distributed in two disjoint radial planes, and wherein said channel is arranged to open out radially into said n recesses such that during one complete revolution of said piston in said body, said piston is n times in each of said fluid-flow configurations.

4. The rotary reciprocating pumping sub-assembly according to claim 1, wherein the sub-assembly includes at least first and second levels, each corresponding, in distinct manner, to a set of two ducts, to a working chamber, to a channel, and to a pair of recesses.

5. The rotary reciprocating pumping sub-assembly according to claim 4, wherein said working chambers present sections that are different.

6. The rotary reciprocating pumping sub-assembly according to claim 1, wherein the sub-assembly includes at least a cam and a guide finger, one carried by said piston, the other by said body, and arranged to co-operate reciprocally so that turning said piston relative to said body causes:
over a first angular portion, said piston to move in axial translation relative to said body in a first direction;
over a second angular portion, said piston to be axially stationary relative to said body;
over a third angular portion, said piston to move in axial translation relative to said body in a second direction; and
over a fourth angular portion, said piston to be axially stationary relative to said body;
said ducts and said recesses being arranged so that said ducts are closed during said second and fourth angular portions.

7. The rotary reciprocating fluid pumping device, in particular for medical use, comprising drive means and a rotary reciprocating sub-assembly for pumping a fluid according to claim 1, and releasable mechanical coupler means for mechanically connecting said drive means to said piston in releasable manner.

8. The rotary reciprocating pumping device according to claim 7, wherein the pumping device includes a rotary reciprocating sub-assembly for pumping a fluid according to claim 5, in that one of said ducts is connected to the inlet for a first fluid, one of said ducts is connected to the inlet for a second fluid,
wherein said rotary reciprocating pumping device includes mixer means for mixing said first fluid discharged by the other of said ducts, and said second fluid discharged by the other of said ducts, the resulting mixture being proportional to the difference in section between said working chambers.
